(19) United States
(12) Patent Application Publication
(10) Pub. No.: US 2018/0263888 A1
(43) Pub. Date: Sep. 20, 2018

(54) CONCENTRATED POLYOLEFIN EMULSIONS AND HAIR CARE COMPOSITIONS CONTAINING THEM

(71) Applicants: Rohm and Haas Company, Collegeville, PA (US); Dow Global Technologies LLC, Midland, MI (US)

(72) Inventors: Nikhil J Fernandes, Philadelphia, PA (US); Jodi A. Thomas, Midland, MI (US); David L. Malotky, Midland, MI (US)

(21) Appl. No.: 15/760,269

(22) PCT Filed: Nov. 14, 2016
(86) PCT No.: PCT/US2016/061787

§ 371 Date: Mar. 15, 2018

Related U.S. Application Data
(60) Provisional application No. 62/266,257, filed on Dec. 11, 2015.

(51) Int. Cl. A61K 8/81; A61K 8/06; A61K 8/92; A61Q 5/12

(52) U.S. Cl. CPC A61K 8/8111; A61K 8/062; A61K 8/92; A61K 2800/594; A61K 2800/30; A61K 2800/48; A61Q 5/12

(57) ABSTRACT

Provided is a concentrated emulsion and hair care compositions containing the concentrated emulsion. The emulsion comprises (a) an internal phase containing (i) a high density polyolefin with a density above 0.90 g/cm3, (ii) a low density polyolefin with a density equal to or below 0.90 g/cm3, and (iii) a cosmetically acceptable hydrocarbon oil; (b) from 0.1 to 15 wt %, based on the total weight of the emulsion, of a surfactant; and (c) balance water as a continuous phase, wherein the high and low density polyolefins have an average melt index greater than 7.

### FIELD

This invention relates generally to compositions that are useful as sensory agents in hair care compositions. The compositions contain a concentrated emulsion of a polyolefin blend.

### BACKGROUND

Personal care products, particularly hair care products such as leave-on conditioners, require a smooth and silky feel on skin to please consumers. In fact, aesthetics are one of the most important factors in consumer satisfaction. Accordingly, the hair care art has developed sensory agents, such as silicone oils, hard particles (such as poly(methyl methacrylate) (PMMA) particles and polyethylene (PE) particles), and silicone elastomer gels in order to impart good aesthetics. However, each of the foregoing is associated with certain drawbacks, like insufficient sensory performance, poor conditioning, stability, and texture, or relatively high cost.

Even where a composition provides desirable performance, it may still be lacking based on how difficult it is to incorporate the composition in a hair care product. For instance, compositions that require high temperatures to processing, or high use amounts to achieve the desired performance, are still disadvantaged, for instance because of the additional costs associated with their use.

Accordingly, there is a continuing need in the art for cost-effective, easily useable, high performance sensory agents, preferably with good manageability, anti-frizz properties, and shine profiles in hair care formulations.

### STATEMENT OF INVENTION

We have now found that polyolefin blends as described herein, which are highly effective sensory agents in hair care compositions, may be prepared as concentrated oil in water emulsions, for instance as high internal phase emulsions where the volume % internal phase is at least 75%. Advantageously, the emulsion is easily incorporated in hair care compositions at low concentrations and eliminates the need for high temperatures to melt the oil gel in the oil phase of such compositions. A hair care composition containing the concentrated emulsion provides improved resistance to hair frizziness in high humidity conditions compared to compositions containing oil gel added separately, and compared to commercial silicone-containing benchmarks.

In one aspect, therefore, there is provided a concentrated emulsion comprising:

(a) from 60 to 95 wt %, based on the total weight of the emulsion, of an internal phase comprising:
(i) a high density polyolefin with a density above 0.90 g/cm³,
(ii) a low density polyolefin with a density equal to or below 0.90 g/cm³, and
(iii) a cosmetically acceptable hydrocarbon oil; and

(b) from 0.1 to 15 wt %, based on the total weight of the emulsion, of a surfactant; and

(c) balance water as a continuous phase,

wherein the high and low density polyolefins have an average melt index greater than 7.

In another aspect, there is provided a hair care composition comprising: (a) a concentrated emulsion as described herein; and (b) a thickener. Preferably, the concentrated emulsion comprises a cationic surfactant.

In a further aspect, there is provided a method for preparing the concentrated emulsion according to the processes described herein.

In a still further aspect, there is provided a method for preparing a hair care composition containing a polyolefin blend and a hair care additive, the method comprising adding the polyolefin blend to the hair care composition in the form of the concentrated emulsion as described herein.

### BRIEF DESCRIPTION OF THE FIGURES

FIG. 1 shows processed images demonstrating the effect of frizz on projected area of a hair tress.

### DETAILED DESCRIPTION

Unless otherwise indicated, numeric ranges, for instance as in “from 2 to 10,” are inclusive of the numbers defining the range (e.g., 2 and 10). Unless otherwise indicated, ratios, percentages, parts, and the like are by weight. As used herein, unless otherwise indicated, the phrase “molecular weight” or Mw refers to the weight average molecular weight as measured in a conventional manner with gel permeation chromatography (GPC) and polyacrylic acid standards. GPC techniques are discussed in detail in Modem Size Exclusion Chromatography, W. W. Yau, J. J. Kirkland, D. D. Bly; Wiley-Interscience, 1979, and in A Guide to Materials Characterization and Chemical Analysis, J. P. Sibilia; VCH, 1988, p. 81-84. Molecular weights are reported herein in units of Daltons. The term “polymer” refers to a polymeric compound prepared by polymerizing monomers, whether of the same or a different type. The generic term “polymer” includes the terms “homopolymer,” “copolymer,” and “terpolymer.” Weight percentages (or wt %) in the composition are percentages of dry or actives weight, i.e., excluding any water that may be present in the composition. Percentages of monomer units in the polymer are percentages of solids or neat monomer weight, i.e., excluding any water present in a polymer emulsion.

“Hair care” relates to compositions to be topically applied to a person, and in particular a person's hair. Examples of hair care compositions include, but are not limited to, shampoos, leave-on and rinse off conditioners, styling gels, and hairsprays. In certain preferred embodiments, the hair care composition is a conditioner, preferably a leave-on hair conditioner. “Cosmetically acceptable” refers to ingredients typically used in hair care compositions, and is intended to underscore that materials that are toxic when present in the amounts typically found in personal care compositions are not contemplated as part of the present invention.

As noted above, in one aspect, the invention provides a concentrated emulsion. The concentrated emulsion is comprised of from 60 to 95 wt % of an internal phase that contains a high density polyolefin with a density (as measured by ASTM D 792) above 0.90 g/cm³, a low density polyolefin with a density equal to or below 0.90 g/cm³, preferably from 0.86 to 0.90 g/cm³, and a cosmetically acceptable hydrocarbon oil.

In certain embodiments, the average melt index (g/10 min, as measured by ASTM D 1238) for the high and low density polyolefins in the polyolefin oil blend is greater than 7, preferably greater than 8, and more preferably greater than 8.5. In certain embodiments, the high density polyolefin has a weight-average molecular weight in a range of from 41,000 to 500,000, preferably 70,000 to 90,000, and more preferably from 75,000 to 85,000. In certain embodiments, the low density polyolefin has a weight-average molecular weight in a range of from 5,000 to 40,000, preferably 10,000 to 30,000, and more preferably from 20,000 to 28,000. In certain preferred embodiments the concentrated emulsion is substantially free of ethylene-acrylic acid copolymer.

Polyolefins for use in the invention are produced with a metallocene catalyst. Metallocene catalysis enables control of the polyolefin properties relating to, for example, crystallinity, polymer chain length, and distribution homogeneity of the polymer chain units. Metallocene catalysis also favors uniformity in polymer chains density and length. Suitable metallocene catalysts include, for example, those described in U.S. Pat. Nos. 4,701,432, 5,322,728, and 5,272,236. In certain embodiments of the present invention, the polyolefins are polyethylenes produced with a metallocene catalyst. Suitable polyethylenes are available from, for example, The Dow Chemical Company under the trademark AFFINITY or ENGAGE (ethylene/octene copolymers), and from Exxon Chemical Company under the trademark EXACT (ethylene/butene copolymers, ethylene/hexene copolymers, or ethylene/butene/hexene terpolymers). In one embodiment, the polyolefin is at least one of ethylene/octene copolymers, ethylene/butene copolymers, ethylene/hexene copolymers, ethylene/propylene or ethylene/butene/hexene terpolymers, preferably an ethylene octene copolymer. In another embodiment, the polyolefin is a propylene/alpha-olefin copolymer. Suitable propylene/alpha-olefin copolymers include, for example, those described in detail in U.S. Pat. Nos. 6,960,635 and 6,525,157. Such propylene/alpha-olefin copolymers are commercially available from The Dow Chemical Company under the trademark VERSIFY, or from ExxonMobil Chemical Company under the trademark VISTAMAXX. Other suitable polyolefins are sold by The Dow Chemical Company under the trademarks AMPLIFY, ATTANE, INFUSE, NORDEL, and VLDPE. Other suitable non-limiting examples of commercially available metallocene catalyzed polyethylenes and the melt index and density of each is as shown in Table 1.

TABLE 1
Specified Metallocene Catalyzed Polyethylenes

| Polyolefin Name | Melt Index | Density |
|---|---|---|
| AFFINITY GA 1950 | 500 | 0.874 |
| AFFINITY PL1840G | 1 | 0.909 |
| AMPLIFY EA 103 | 21 | 0.930 |
| AMPLIFY GR 202 | 8 | 0.930 |
| ATTANE 4203 | 0.8 | 0.905 |
| ATTANE 4404G | 4 | 0.904 |
| ENGAGE 8100 | 1 | 0.870 |
| ENGAGE 8130 | 13 | 0.863 |
| ENGAGE 8200 | 5 | 0.870 |
| ENGAGE 8402 | 30 | 0.902 |
| INFUSE D9807 | 15 | 0.866 |
| LDPE 4016 | 16 | 0.916 |
| LDPE 640I | 2 | 0.920 |
| LDPE 955I | 35 | 0.923 |
| VERSIFY 2200 | 2 | 0.876 |
| VERSIFY 3200 | 8 | 0.876 |
| VERSIFY 4200 | 25 | 0.876 |

In certain preferred embodiments the inventive compositions described herein are substantially free of EAA. “Substantially free” in this context means less than 3 weight %, preferably less than 1 weight %, more preferably less than 0.1 weight %, and even more preferably zero weight percent present in the composition.

In one embodiment, the high density polyolefin is present in the internal phase in an amount of from 5 to 30 weight %, preferably from 2 to 16 weight %, of solids by weight of the internal phase. In one embodiment, the low density polyolefin is present in an amount of from 5 to 20 weight %, preferably from 5 to 15 weight %, of solids by weight of the internal phase. In one embodiment, the ratio of polyolefin with a density above 0.90 g/cm³ to the polyolefin with a density equal to or below 0.90 g/cm³ is between 1:95 and 95:1, preferably between 10:50 and 60:10, and more preferably between 10:40 and 40:10. In certain preferred embodiments, the ratio is 1:1, 1.5:1, 2:1, or 3:1.

A variety of cosmetically acceptable hydrocarbon oils are suitable for use in the present invention, and are selected from various carbon chain length oils. In certain embodiments, the hydrocarbon oils include, but are not limited to, C₁₄-C₂₂ hydrocarbon oils. In certain embodiments, the hydrocarbon oil is less than fourteen carbons in length. In certain embodiments, the hydrocarbon oil is greater than twenty-two carbons in length. Suitable hydrocarbon oils include, for example, those sold under the trademarks LILAC, GEMSEAL 25, GEMSEAL 40, PERMETHYL 101A, PERMETHYL 99A, SILKFLO 364 NF, SILKFLO 366 NF, FANCOL POLYISO 200-CG, FANCOL POLYISO 300-CG, FANCOL POLYISO 450-CG, FANCOL POLYISO 800-CG, PANALANE L-14E, PURESYN 2, PURESYN 4, or RITADECENE 20. A preferred hydrocarbon oil is lilac white oil. In certain embodiments, the hydrocarbon oil is present in an amount of from 35 to 90 weight %, preferably 65 to 85 weight %, by weight of the internal phase.

The concentrated emulsion contains a surfactant. The surfactant may be cationic, nonionic, anionic, or combinations thereof. The surfactant is typically present in the concentrated emulsion in an amount ranging from 0.1 to 15 wt %, preferably 2 to 8 wt %, by weight of the concentrated emulsion. In a preferred embodiment, the surfactant comprises a cationic surfactant or a mixture of a cationic surfactant and a nonionic surfactant.

Cationic surfactants suitable for use in the concentrated emulsion include, for example, quaternary ammonium salt surfactants. Suitable quaternary ammonium salt surfactants include, for instance, dialkyldimethylammonium salt surfactants, alkylbenzyldimethyl-ammonium salt surfactants, alkyltrimethylammonium salt surfactants, and alkylpyridinium halide surfactants. Suitable quaternary ammonium salt surfactants have corresponding anions. Suitable corresponding anions include, for example, halide ions (such as, for example, chloride ions), methyl sulfate ions, other anions, and mixtures thereof. Specific examples of cationic surfactants include, without limitation, behenyltrimethylammonium chloride, or cetrimonium chloride. Cetrimonium chloride is preferred. Mixtures of cationic surfactants may be used.

Anionic surfactants that may suitable by used in the concentrated emulsion include, without limitation, C11-C18 alkyl benzene sulfonates and primary or branched-chain C10-C20 alkyl sulfates, unsaturated sulfates such as oleyl sulfate, the C10-C18 alkyl alkoxy sulfates, particularly those comprising 1-7 ethoxy groups, C10-C18 alkyl alkoxy carboxylates, particularly those comprising 1-5 ethoxy groups, the C10-C18 glycerol ethers, the C10-C18 alkyl polyglycosides and their corresponding sulfated polyglycosides, and C12-C18 alpha-sulfonated fatty acid esters. Other useful anionic surfactants include water-soluble salts, particularly the alkali metal, ammonium and alkylolammonium salts, such as monoethanolammonium or triethanolammonium salts, of organic sulfuric reaction products having in their molecular structure an alkyl group containing from about 10 to about 20 carbon atoms and a sulfonic acid or sulfuric acid ester group. Other anionic surfactants useful herein are the water-soluble salts of alkyl phenol ethylene oxide ether sulfates and water-soluble salts of esters of alpha-sulfonated fatty acids. Anionic surfactants based on fatty acids may be used and include saturated and/or unsaturated fatty acids obtained from natural sources or synthetically prepared. Examples of suitable fatty acids include, but are not limited to, capric, lauric, myristic, palmitic, stearic, arachidic, and behenic acid. Other fatty acids include palmitoleic, oleic, linoleic, linolenic, and ricinoleic acid. Examples of particularly preferred surfactants are fatty acid salts, sulfonates or quaternary ammonium salts, and especially sodium lauryl sulfate (SLS) or sodium laureth sulfate (SLES).

Nonionic surfactants that may be used in the concentrated emulsion of the invention include, for example, polyoxyalkylene surfactants, polyalkylene glycol esters, polyoxyethylene derivatives of fatty acid esters of polyhydric alcohols, fatty acid esters of polyalkoxylated polyhydric alcohols, polyalkoxylated natural fats and oils, polyalkylene oxide block copolymers, alkyl polyglucosides, sucrose esters, and mixtures thereof. Among the suitable polyoxyalkylene surfactants, some suitable examples are polyoxyethylene surfactants, including, for example, alcohol alkoxylates, alkylphenol alkoxylates, and mixtures thereof. Suitable alcohol alkoxylates include, for example, alcohol ethoxylates and alcohol propoxylates. In some embodiments, one or more alcohol ethoxylate is used. Specific examples of nonionic surfactants include, without limitation, laureth-23, ceteth-20, or steareth-100. The concentrated emulsion of the invention contains water as a continuous phase. The water generally comprises the balance of the concentrated emulsion, to bring the emulsion to 100%, after the amounts of the other ingredients have been selected. In some embodiments, the amount of water in the emulsion is at least 5 wt %, alternatively at least 10 wt %, and up to 40 wt %, alternatively up to 30 wt %, by weight of the concentrated emulsion. As is apparent, the emulsion of the invention contains the internal, oil phase, at a high concentration. In some embodiments, the concentrated emulsion may be considered a high internal phase emulsion (HIPE) containing, for instance, at least 75 wt % of the internal phase, by weight of the emulsion.

The emulsion of the invention may contain optional ingredients, including additional surfactants, such as zwitterionic surfactants, and preservatives such as benzoic acid, sorbic acid, or phenoxyethanol.

The concentrated emulsion may be prepared by a variety of methods, including batch and continuous methods well known in the art. In a preferred continuous method (described generally by Pate et al in U.S. Pat. No. 5,539,021, column 3, line 15 to column 6, line 27, which is incorporated herein by reference), a stream containing the continuous aqueous phase is flowed through a first conduit and merged continuously with a stream of the disperse internal phase that is flowed through a second conduit. The streams are merged into a disperser in the presence of one or more surfactants. The surfactants can be added to either stream, or as a separate stream. Additional details can be found, for instance, in U.S. Pat. No. 6,783,766, which is incorporated herein by reference.

In another aspect, the invention provides a hair care composition comprising a concentrated emulsion as described above and a thickener. Preferably, the concentrated emulsion of the hair care composition contains a cationic surfactant. In certain embodiments, the concentrated emulsion is present in an amount of from 0.1 to 10 weight %, preferably 0.1 to 5 weight %, and more preferably 0.5 to 2.5 weight %, by weight of the hair care composition.

Thickeners are substances that increase the viscosity of media preferably without substantially modifying the media's other properties. Suitable thickeners include, for example, polysaccharides (e.g., xanthan gum, guar gum, starch, and vegetable gum) and cellulosic polymers (e.g., carboxymethyl cellulose (CMC), hydroxymethyl cellulose (HMC), and hydroxypropyl methyl cellulose (HPMC)). Certain preferred thickeners include, for example, hydrophobically modified cross-linked acrylate copolymers (e.g., those sold by Lubrizol under the trademark CARBOPOL ULTREZ 21). In certain embodiments, the thickener is present in an amount of from 0.1 to 1.0 weight %, preferably from 0.2 to 0.7 weight %, and more preferably from 0.3 to 0.5 weight %, by weight of the hair care composition.

Humectants are optionally added to the composition to prevent the loss of moisture. Suitable humectants include, for example, glycerin, sorbitol, monoglycerides, lecithins, glycolipids, fatty alcohols, fatty acids, polysaccharides, sorbitan esters, polysorbates (e.g., Polysorbate 20, Polysorbate 40, Polysorbate 60, and Polysorbate 80), diols (e.g., propylene glycol), diol analogs, triols, triol analogs, polymeric polyols, and mixtures thereof. In certain embodiments, the humectant is present in an amount of from 1 to 20 weight %, preferably 2 to 15 weight %, and more preferably 5 to 10 weight %, by weight of the hair care composition.

The hair care composition may also contain water, in addition to the water provided by the concentrated emulsion. The water generally comprises the balance of the hair care composition, to bring the composition to 100%, after the amounts of the other required and optional ingredients have been selected. In some embodiments, the amount of water may, for instance, range from 85 to 95 weight %, preferably from 60 to 80 weight %, and more preferably from 50 to 70 weight %, by weight of the hair care composition.

In certain embodiments, the hair care composition contains excipients, such as additional emollients (e.g., hydrocarbon oils, esters, natural oils, or silicones), waxes, sensory modifiers, lubricants, preservatives (e.g., benzoic acid), antioxidants (e.g., butylated hydroxytoluene), chelating agents, antimicrobials, pH adjusting agents/buffers/neutralizing agents, sunscreen actives, vitamins, proteins/amino acids, plant extracts, natural ingredients, bio-actives, fragrances/perfumes, penetrants, polymers/resins/hair fixatives/film formers, surfactants/detergents/emulsifiers/opacifying agents, volatiles/propellants/solvents/carriers, liquid vehicles/solvents/carriers, salts, anti-static agents, anti-frizz agents, antidandruff agents, hair waving/straightening agents, absorbents, colorants, hard particles, conditioning agents, and other silicones.

Hair care compositions of the invention that contain ingredients other than the concentrated emulsion may generally be prepared by creating the formulation in the manner appropriate for the thickener. Hair compositions of the invention that contain a thickener may be prepared by either adding the thickener to a diluted dispersion of the concentrate, or by adding the concentrated emulsion to a thickened aqueous formulation.

Some embodiments of the invention will now be described in detail in the following Examples.

### EXAMPLES

### Example 1. Preparation of Cationic Concentrated Emulsion

A polyolefin gel was synthesized in an oil jacketed five gallon batch mixer (Model #VME-12 available from Fryma Maschinen AG, Switzerland) equipped with a sweep mixing blade. The mixer was loaded with 4788 g of isohexadecane (Permethyl 101A from Presperse) and the sweep mixing blade was turned on at a speed of 60 rpm. 456 g each of AFFINITY PL1840G and AFFINITY GA1950 were weighed out and blended together. This blend was slowly added to the mixing isohexadecane, after which the oil jacket was used to heat the mixture to an internal temperature of 117° C. under continued mixing. This heat up step took 100 minutes. Once the material reached the target temperature for 117° C., mixing was continued for an additional 60 minutes, after which the oil jacket temperature setpoint was reduced to 66° C., causing the mixture to cool down under continued mixing. After an additional 150 minutes the internal temperature of the mixer had dropped below 70° C. and the olefin gel was unloaded from the mixer for later use.

An internal phase of a cationic concentrated emulsion was prepared by combining 3% steareth-100, 3% laureth-23, and 0.75% benzoic acid with 93.25% of the polyolefin gel described above and heating this mixture to 100° C. The phase was then mixed for 1 minute with a propeller mixer to form a uniform opaque phase. This phase was loaded into a Nordson Altablue 4TT hot melter where the reservoir and delivery line have both been set to 110° C. The phase was then pumped at a rate of 14 g/min into a two inch diameter rotor stator mixer heated to 110° C. and spinning at 850 rpm. The internal phase was merged at the mixer with a separate deionized water stream flowing at 1.0 ml/min and a second aqueous stream of 30% active cetrimonium chloride flowing at 0.5 ml/min. Both aqueous streams were fed by 500 ml Isco syringe pumps. The resultant polyolefin gel emulsion has a volume mean particle size of 2.7 microns and flowed into a second two inch Oakes mixer heated to 110° C. and spinning at 450 rpm where it was combined with an additional deionized water stream flowing at 2 ml/min to dilute the cationic concentrated emulsion down to 80% internal phase. The cationic concentrated emulsion then passed through an exit tubing set to 90° C. and a backpressure regulator set to 50 psi, which keeps the water in the process liquid at all times.

### Example 2. Preparation of Nonionic Concentrated Emulsion

An internal phase of a nonionic concentrated emulsion was prepared by combining 3% ceteth-20, 3% laureth-23, and 0.75% benzoic acid with 93.25% of the polyolefin gel described in Example 1 above and heating this mixture to 100° C. The phase was then mixed for 1 minute with a propeller mixer to form a uniform opaque phase. This phase was loaded into a Nordson Altablue 4TT hot melter where the reservoir and delivery line have both been set to 110° C. The phase was then pumped at a rate of 20 g/min into a two inch diameter rotor stator mixer heated to 110° C. and spinning at 850 rpm. The internal phase was merged at the mixer with a separate deionized water stream flowing at 1.75 ml/min fed by a 500 ml Isco syringe pumps. The resultant olefin gel emulsion had a volume mean particle size of 1.3 microns and flowed into a second two inch Oakes mixer heated to 110° C. and spinning at 450 rpm where it was combined with an additional deionized water stream flowing at 2.5 ml/min to dilute the nonionic concentrated emulsion down to 82.5% internal phase. The nonionic concentrated emulsion then passed through an exit tubing set to 90° C. and a backpressure regulator set to 50 psi, which keeps the water in the process liquid at all times.

### Example 3. Preparation of Anionic Concentrated Emulsion

An anionic concentrated emulsion concentrate used the polyolefin gel described in Example 1 as the internal phase without any further additions. The polyolefin gel was heated to 110° C. until it became a clear uniform fluid, and was loaded into a Nordson Altablue 4TT hot melter where the reservoir and delivery line have both been set to 110° C. The phase was then pumped at a rate of 14 g/min into a two inch diameter rotor stator mixer heated to 110° C. and spinning at 900 rpm. The internal phase was merged at the mixer with a separate deionized water stream flowing at 0.8 ml/min and a second aqueous stream consisting entirely of EMPICOL ESB70 (70% active sodium laureth sulfate in water). Both aqueous streams were fed by 500 ml Isco syringe pumps. The resultant olefin gel emulsion had a volume mean particle size of 0.5 microns and flowed into a second two inch Oakes mixer heated to 110° C. and spinning at 450 rpm where it was combined with an additional deionized water stream flowing at 3 ml/min to dilute the anionic concentrated emulsion down to 78% internal phase. The anionic concentrated emulsion then passes through an exit tubing set to 90° C. and a backpressure regulator set to 50 psi, which keeps the water in the process liquid at all times.

### Example 4. Preparation of a Leave-on Hair Conditioner Including a Concentrated Emulsion

A leave-on hair conditioner including the cationic concentrated emulsion prepared in Example 1 above is prepared according to the formulation in Table 2.

TABLE 2
Example Leave-On Hair Conditioner Formulation
Including cationic concentrated emulsion

| Phase | Ingredient | INCI | g Added |
|---|---|---|---|
| A | Water | Deionized water | 89.85 |
| B | Cetearyl Alcohol | Cetearyl Alcohol (a | 6.0 |
| | | thickener) | |
| B | Cetrimonium | Cetrimonium Chloride | 0.20 |
| | Chloride | | |
| B | Ceteareth 20 | Ceteareth 20 (a thickener) | 0.30 |
| B | Glycerin | Glycerin | 1.0 |
| B | PG USP | Propylene glycol | 0.50 |
| B | BHT | Butylated hydroxytoluene | 0.05 |
| | | (an antioxidant) | |
| C | Kathon CG | Methylchloroisothiazolinone | 0.10 |
| | | and Methylisothiazolinone | |
| D | UCON AP | PPG-14 Butyl Ether (an | 1.0 |
| | | emollient) | |
| E | Example 1 emulsion | — | 2.0 |
| | Total | | 100 |

### Procedure:

1. Heat water to 80-90° C. (phase A)
2. Add all the ingredients in Phase B together into a separate container and heat to 80-90° C.
3. When up to temperature and oil phase is melted, pour phase B into phase A while stirring at 800 rpm.
4. Stir for 1 hour, take off heat, and continue stirring.
5. When formulation has cooled to 45° C., add Kathon CG and stir for 5 minutes.
6. When the formulation has reached room temperature, add 1 g of UCON AP with continuous stirring.
7. Add 2 g of the Example 1 emulsion and maintain stirring for 5 minutes

### Example 5 (Comparative)

In order to create a control sample, the same procedure was followed, except that the Example 1 emulsion was omitted, and instead 2 g of the polyolefin gel from Example 1 was melted into the oil phase (Step 2).

### Example 6 (Comparative)

An additional control was the formulation as listed above, but omitting any polyolefin oil gel altogether, and adding 2 g of water instead.

### Example 7

The procedure as in Example 4 was followed, but 2 g of Example 2 (containing nonionic surfactant) was used in place of Example 1.

### Example 8

The procedure as in Example 4 was followed, but 2 g of Example 3 (containing anionic surfactant) was used in place of Example 1.

Samples were tested as follows: Briefly, 5 g of round-bound hair (Brazilian Wavy, International Hair Importers, Inc.) was rinsed for 30 seconds. Then 0.5 g of a 10% w/w Tergitol 15-S-9 (a surfactant) solution was massaged into the hair for 30 seconds, and the tress was rinsed for another 30 seconds. Finally, 0.5 g of the leave-in conditioner formulation was added, and massaged in for 30 seconds. The water used for rinsing was softened to a hardness of 80 ppm and heated to 38° C.

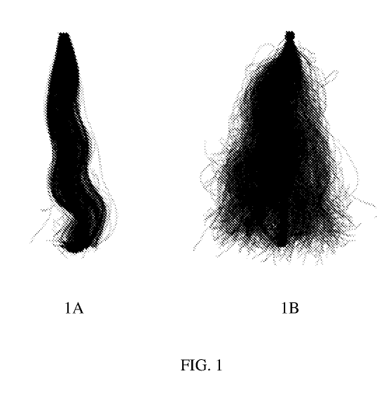

Samples were imaged before treatment, after treatment and drying in a constant temperature environment (40% relative humidity (RH), 24° C.), and after exposure to the humidity chamber (90% RH, 40° C.). The samples were imaged against a white backlight using a Canon T5i DSLR. The images were processed in ImageJ to get the total projected area of each tress. FIG. 1 shows examples of two processed images, with low frizz (1A) and high fizz (1B).

The measurements were repeated on a total of 3 tresses per formulation under each condition The percentage change in area before and after exposure was calculated, according to the formula

$$\% change = \frac{Final Area - Initial Area}{Initial Area} \times 100.$$

The average of the measurements are shown in Table 3.

TABLE 3

| | % Change in Area | % Change in area |
|---|---|---|
| | when exposed to | when exposed to |
| Sample | 40% RH, 24° C. | 90% RH, 40° C. |
| Example 4 (inventive) | 23.6% | 51.9% |
| Example 5 (comparative) | 63.2% | 86.7% |
| Example 6 (comparative) | 33.2% | 102% |
| Example 7 (inventive) | 34% | 133% |
| Example 8 (inventive) | 23% | 107.5% |
| Untreated tress (comparative) | 125% | 145% |

The change in area on exposure to humidity was greatest for the untreated sample (145%) and the lowest for the sample treated with the inventive Example 1 cationic concentrated emulsion (cationic HIPE). The inventive emulsion also showed significant improvement over example 5, which contains olefin gel added separately, example 6, which is an identical conditioner formulation without olefin gel in any form, example 7, which contains a nonionic emulsion of the olefin gel, and example 8, which contains an anionic emulsion of the olefin gel.

What is claimed is:

1. A concentrated emulsion comprising:
(a) from 60 to 95 wt %, based on the total weight of the emulsion, of an internal phase comprising:
(i) a high density polyolefin with a density above 0.90 g/cm³,
(ii) a low density polyolefin with a density equal to or below 0.90 g/cm³, and
(iii) a cosmetically acceptable hydrocarbon oil; and
(b) from 0.1 to 15 wt %, based on the total weight of the emulsion, of a surfactant; and
(c) balance water as a continuous phase,
wherein the high and low density polyolefins have an average melt index greater than 7.

2. The concentrated emulsion of claim 1, wherein the emulsion is substantially free of ethylene-acrylic acid copolymer.

3. The concentrated emulsion of any one of claims 1-2, wherein the weight ratio of the high density polyolefin to the low density polyolefin is from 2:1 to 1:1.

4. The concentrated emulsion of any one of claims 1-3, wherein the cosmetically acceptable hydrocarbon oil is a C₁₄-C₂₂ hydrocarbon oil.

5. The concentrated emulsion of any one of claims 1-4, wherein the internal phase comprises: from 5 to 30 wt % of the high density polyolefin, from 5 to 20 wt % of the low density polyolefin, and balance the cosmetically acceptable hydrocarbon oil, each based on the total weight of the internal phase.

6. A hair care composition comprising:
(a) the concentrated emulsion of any one of claims 1-5; and
(b) a thickener.

7. The hair care composition of claim 6 wherein the concentrated emulsion comprises a cationic surfactant.

8. A method of making a hair care composition that contains a polyolefin blend and a hair care additive, the method comprising: adding the polyolefin blend to the hair care composition in the form of the concentrated emulsion of any one of claims 1-5.
